(19) United States
(12) Patent Application Publication
(10) Pub. No.: US 2015/0173662 A1
(43) Pub. Date: Jun. 25, 2015

(54) SYSTEMS AND METHODS FOR MINIMALLY-INVASIVE ARTERIAL BLOOD GAS MEASUREMENT

(71) Applicant: KONINKLIJKIE PHILIPS N.V., EINDHOVEN (NL)

(72) Inventors: Shrutin Ulman, Bangalore (IN); Sanjay Jayavanth, Bangalore (IN)

(21) Appl. No.: 14/414,799

(22) PCT Filed: Jul. 30, 2013
(86) PCT No.: PCT/IB2013/056264

§ 371 Date: Jan. 14, 2015

(30) Foreign Application Priority Data
Aug. 8, 2012 (IN) 3256/CHE/2012

(51) Int. Cl. A61B 5/157; A61B 5/145; A61B 5/00; A61B 5/15

(52) U.S. Cl. CPC A61B 5/157; A61B 5/15003; A61B 5/14542; A61B 5/4836; A61B 5/150251

(57) ABSTRACT

Provided are systems and methods for minimally invasive arterial blood gas measurements. Blood samples are collected using capillary microstructures that minimize patient discomfort and collect samples in a manner such that the samples are not exposed to an environment outside of the sample collection portion. One or more characteristics of the blood sample are then calculated and used to derive one or more arterial blood gas measurements for the sample.

The present disclosure pertains to systems and methods for minimally invasive blood gas measurement.

Arterial blood gas (ABG) measurement is often an important tool in the care of patients on ventilators in intensive care units (ICUs). Conventional methods of ABG measurement involve the puncturing of an artery and obtaining a blood sample therefrom. This can be a painful procedure, and the logistics of obtaining such a sample often result in exposing the sample to air or other environmental elements that cause errors in ABG measurements. Conventional ABG measurements are also typically sent to remote laboratories for processing, which can introduce errors in sample transport/transfer, handling of samples by multiple persons, and other reasons. Remote laboratory handling also introduces delay in the receipt of results.

Other problems may also exist with conventional methods of ABG measurement.

Accordingly, it is an object of one or more embodiments of the present invention to provide a system for providing arterial blood gas measurements comprising: a sample collection portion positioned in contact with a tissue of the patient such that a blood sample travels from the tissue of the patient into the sample collection portion without being exposed to an environment outside of the sample collection portion; one or more analysis portions in fluid communication with the sample collection portion, wherein each of the one or more analysis portions analyze one or more characteristics of the blood sample; and at least one processor configured to: receive the one or more characteristics of the blood sample and calculate one or more arterial blood gas measurements using the one or more characteristics.

It is yet another aspect of one or more embodiments of the present invention to provide a method for providing arterial blood gas measurements, comprising: positioning a sample collection portion in contact with a tissue of the patient such that a blood sample travels from the tissue of the patient into the sample collection portion without being exposed to an environment outside of the sample collection portion, and wherein the blood sample travels to one or more analysis portions in fluid communication with the sample collection portion, each of the one or more analysis portions analyzing one or more characteristics of the blood sample; receiving at one or more processors of a computational portion, the one or more characteristics of the blood sample; and calculating one or more arterial blood gas measurements using the one or more characteristics.

It is yet another aspect of one or more embodiments of the present invention to provide a system for providing arterial blood gas measurements, comprising: sample collection means positioned in contact with a tissue of the patient such that a blood sample travels from the tissue of the patient into the sample collection means without being exposed to an environment outside of the sample collection means; one or more analysis means in fluid communication with the sample collection means for analyzing one or more characteristics of the blood sample; processing means configured to: receive the one or more characteristics of the blood sample, and calculate one or more arterial blood gas measurements using the one or more characteristics.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

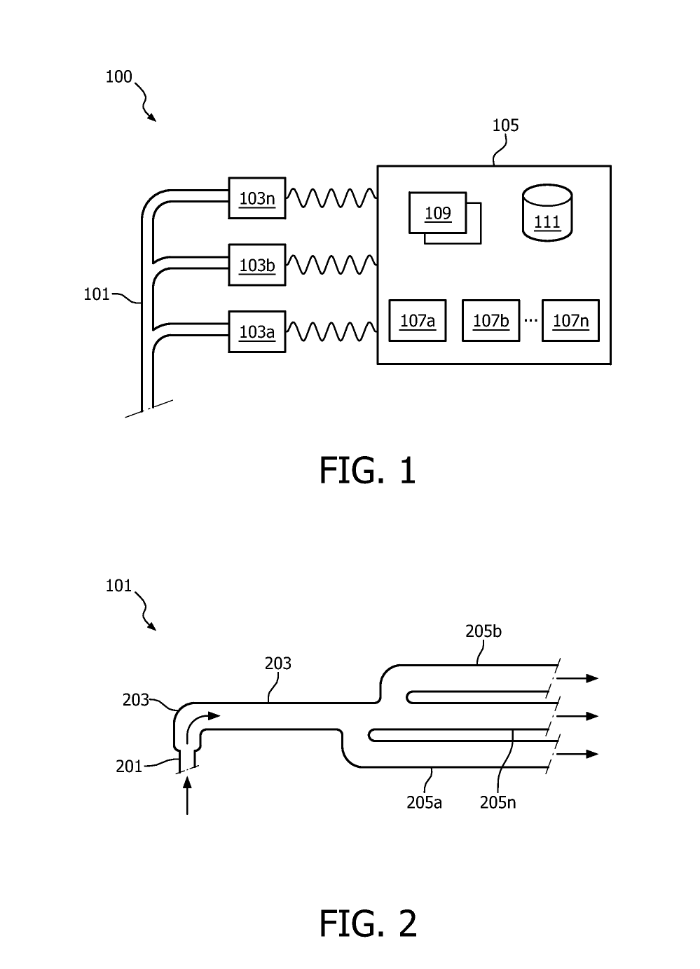

FIG. 1 is an example of a system for minimally invasive arterial blood gas measurements, according to various embodiments of the invention.

FIG. 2 is an example of a collection portion of a system for minimally invasive arterial blood gas measurements, according to various embodiments of the invention.

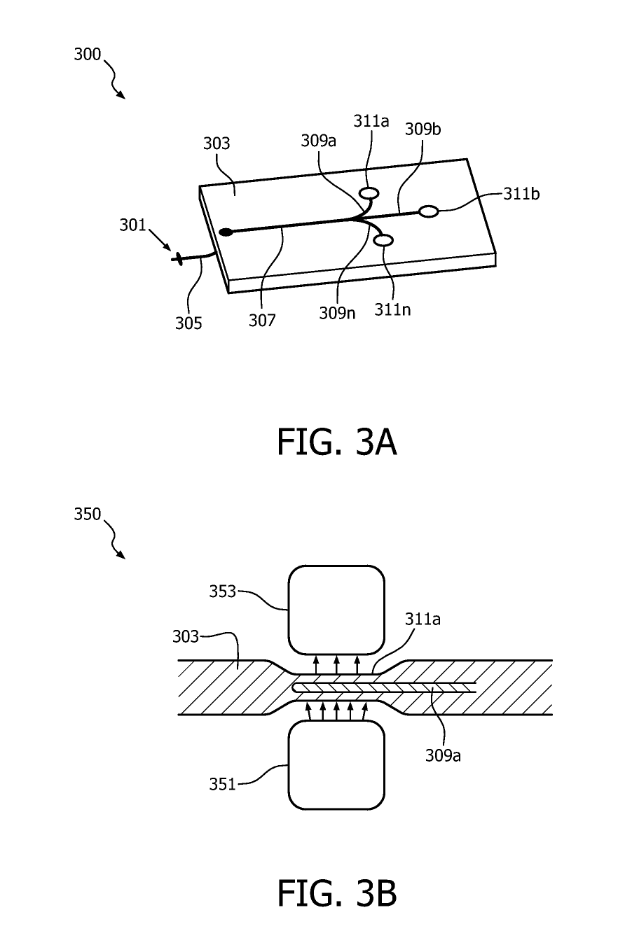

FIG. 3A is an example of a sample collection portion of a system for minimally invasive arterial blood gas measurements, according to various embodiments of the invention.

FIG. 3B is an example of an analysis portion for a system for minimally invasive arterial blood gas measurements, according to various embodiments of the invention.

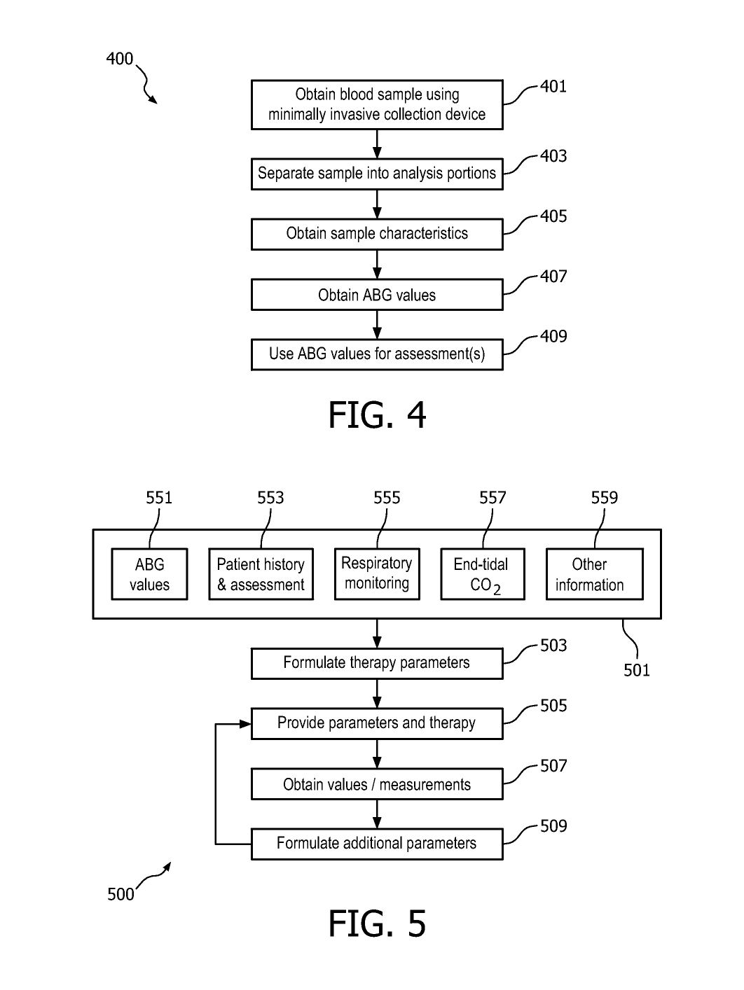

FIG. 4 is an example of a method for minimally invasive arterial blood gas measurements, according to various embodiments of the invention.

FIG. 5 is an example of a method for use of arterial blood gas measurements in a closed loop respiratory therapy, according to various embodiments of the invention.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The systems and methods described herein enable arterial blood gas (ABG) measurements using minimally invasive techniques. The systems and methods described herein may circumvent problems associated with conventional ABG measurement techniques. In some embodiments, the systems and methods described herein may derive or estimate ABG values from blood taken from other parts of the body. This may enable the use of minimally invasive collection techniques and collection devices that minimize or eliminate exposure of samples to the air or other foreign environments. Furthermore, in the techniques and apparatus described herein, ABG measurements may be obtained in a point of contact (POC) environment rather than transferring samples to a remote laboratory, further providing solutions to conventional techniques.

In some embodiments, systems for minimally invasive measurement of ABG values are provided. FIG. 1 illustrates a system 100, which is an example of a system for minimally invasive measurement of ABG and/or other blood-related values. In some embodiments, system 100 may include a sample collection portion 101, one or more analysis portions 103a-103n, a computational system 105, and/or other elements.

In some embodiments, sample collection portion 101 may be or include a minimally invasive collection apparatus. FIG. 2 illustrates an example of sample collection portion 101. In some embodiments, sample collection portion 101 may be a microtubule structure having a total volume of 2-4 μl. In some embodiments, microtubules of sample collection portion 101 may have a diameter of 10 μm. Other dimensions or volumes may be used for collection portion 101.

In some embodiments, sample collection portion 101 may include a tissue engagement portion 201 that contacts the tissue of a patient and enables blood from said tissue to flow into sample collection portion 101. In some embodiments, tissue engagement portion may include a sharp-ended needle that is able to puncture through or “prick” a patient's skin. For example, in some instances, a needle portion of tissue engagement portion 201 may penetrate into tissue having capillaries, therefore enabling capillary blood to flow into sample collection portion 101. In some instances, a needle of tissue engagement portion 201 may penetrate into tissue having a vein, therefore enabling venous blood to flow into sample collection portion 101. In some embodiments, tissue engagement portion 201 may be a hollow metal needle or cannula having a diameter (e.g., 3-4 μm) that minimally damages the tissue through which it punctures (including vascular walls). Tissue engagement portion 201 and sample collection portion 101 may be sized so that a small amount of blood is collected for analysis (e.g., as low as 4 μl). This small sample size enables collection of blood for ABG measurement to be done in a less-painful manner than conventional techniques.

Sample collection portion 101 may also include a main conduit portion 203, which may be a microtube that receives blood from tissue engagement portion 201. In some embodiments, main conduit 203 may be a glass or polymer microtube. In some embodiments, main conduit 203 may be of a diameter such that one of the factors contributing to the flow of blood therethrough is capillary action (other motive forces for blood through sample collection portion 101 may include, for example, the pressure of blood within the tissue of the patient). Accordingly, blood collected into main conduit may continue to flow further into sample collection portion 101. In some embodiments, main conduit 203 may be lcm long (or longer) and may have a diameter of 10 μm. Other dimensions may be used.

In some embodiments, sample collection portion 101 may include a plurality of analyte separation portions 205a-205n. In some embodiments, analyte separation portions 205a-205n and main conduit 203 may be 1 cm in length (or longer) and 10 μm in diameter. Other dimensions may be used. Each analyte separation portion 205 may carry blood from main conduit 203 to a mechanism for measuring/determining a characteristic of the blood (see e.g., analysis portions 103a-103n of FIG. 1). For example, one analyte separation portion 205 may carry blood to components for measuring CO₂ concentration in the blood. Another analyte separation portion 205 may carry blood to components that measure the O₂ concentration in the blood. Another analyte separation portion 205 may carry blood to components that measure the pH of the blood. Other analyte separation portions 205 may be used to carry blood to other analysis components for measuring other characteristics. In some embodiments, each of analyte separation portions 205a-205n may be or include a glass or polymer microtube. Accordingly, in some embodiments, the blood may be carried through analyte separation portions via capillary action. Use of multiple analyte separation portions 205a-205n enables measurement of multiple characteristics using a single “prick” to the tissue of a patient, which further reduces the pain experienced by the patient when obtaining ABG values. This may be especially valuable in neonatal intensive care unit (NICU) and other intensive care units (ICU) wherein patient health can be fragile.

In some embodiments, main conduit 203 and/or other parts of sample collection portion 101 may be filled with one or more substances (e.g., nitrogen or other inert gases) so as to provide a non-reactive environment in which to collect blood (e.g., free from oxygen, air, or other reactive substances). In some embodiments, a vacuum may be created in main conduit 203 and/or other parts of sample collection portion 101 so that incoming blood samples are not exposed to oxygen, air, or other substances that may effect ABG or other blood measurements. In some embodiments, the dimensions of sample collection portion (e.g., the use of microtubes) may have such a small volume of empty space prior to collecting a sample that exposure of a blood sample to error-causing substances (e.g., oxygen, air, or other reactive substances) is de-minimis.

One or more analysis portions 103a-103n of system 100 may each include components that measure certain characteristics of a blood sample. For example, an analysis portion 103 for measuring CO₂ concentration in the blood sample may include a spectrograph that may include a light emitter and light detection portions that are positioned so as to emit light (or other EM radiation) through the blood sample (e.g., contained in a microtubule or microchannel portion of an analysis portion 103) and detect any light absorbed by the blood (indicating concentration of CO₂ in the blood). Similar components may be used in an analysis portion 103 for measuring O₂ concentration in the blood. One or more analysis portions 103a-103n may also include components for measuring: a pH of a blood sample (e.g., a pH nanoelectrode), glucose-6-phosphate dehydrogenase (G6PD) deficiency (measured using, for example, a spectrograph), jaundice measurement (e.g., bilirubin levels, measured using for example, a spectrograph), and/or other measurements.

Computational system 105 may be or include one or more computing devices (e.g., specialty computing systems, desktop computers, personal computers, mobile computing devices, tablet computing devices, smartphones, or other computing devices) having one or more processors 109 (e.g., microprocessors), memory devices 111 (e.g., hard disk, RAM, eeprom, etc.), input/output components, and/or other computing components for performing the features and functions described herein (and/or other features and functions). In some embodiments, computational system 105 may include one or more modules 107a-107n which comprise instructions that, when executed, cause one or more processors 109 of computational system 105 to perform the various features and functions described herein. For example, in some embodiments, one or more of modules 107a-107n may enable calculation and/or receipt of data relating to characteristics of a blood sample (CO₂ levels, O₂ levels, pH, etc.), derivation or other determination of ABG values (e.g., CO₂ levels, O₂ levels, pH, etc.) from characteristics of non-arterial blood samples, providing patient heath/pathology evaluations using ABG values and/or other information, calculation of ventilation or other respiratory therapy parameters using arterial blood values and/or other values, and/or for performing other calculations/determinations.

In some embodiments, sample collection and analysis portions of systems for minimally invasive measurement of ABG and/or other blood-related values may have different configurations. FIGS. 3A and 3B illustrate sample collection and analysis portions of an example system for minimally invasive measurement of ABG and/or other blood-related values. FIG. 3A illustrates sample collection and analyte separation portion 300, which includes a tissue engagement portion 301 that is connected to an analyte separation chip 303 via a connection portion 305. Patent engagement portion may be or include a microfluidic needle or cannula that may puncture or “prick” the tissue of a patient and collect a blood sample. Connection portion 305 may be or include a microfluidic tube that transports the blood sample from tissue engagement portion 301 to analyte separation chip 303. In some embodiments, a needle comprising tissue engagement portion 301 may be about 3-4 μm in diameter and connection portion 305 may be about 10 μm in diameter. Other dimensions may be used.

In some embodiments, analyte separation chip 303 may be or include a planar chip or other object made from silicon, glass, polymer plastic, or other material and having one or more microchannels etched or embedded therein. In some embodiments, analyte separation chip 303 may be or include a chip having dimensions of about 2 cm×4 cm. The one or more microchannels may include a main microchannel 307 that splits into one or more branch channels 309a-309n. In some embodiments, main microchannel 307 and branch channels 309a-309n may each be about 1 cm in length with a diameter of about 10 μm. Each of branch channels 309a-309n may terminate at an analysis portion 311 (see e.g., 311a-311n). In some embodiments, the diameter of analysis portions 311 may be about 50 μm. Other dimensions may be used.

A blood sample may be introduced into main microchannel 307 from connection portion 305. Through capillary action (or other motive force), the blood sample may move into each of branch channels 309a-309n, and into their respective analysis portions 311. One or more characteristics of the blood sample may then be measured in each analysis portion 311. For example, in some embodiments, an analysis portion 311 may include a window or other area that enables light to be transmitted through the blood sample therein. In some embodiments, analysis portions 311 may include one or more microtubules or microchannels (e.g., portions of branch channels 309 that are within a window or other area of an analysis portion 311 enabling light to be transmitted through a blood sample). FIG. 3B illustrates an analysis apparatus 350, which may include or be part of a spectrograph, wherein a light (or other EM radiation) source 351 is positioned so as to direct light (or other EM radiation) onto a blood sample at an analysis window 311a. A radiation detector 353 is positioned opposite light source 351 so as to detect the light that is transmitted through the blood sample in analysis window 311a. From the radiation that is absorbed by the blood sample in analysis window 311a, certain characteristics of the blood sample (e.g., O₂, CO₂, etc.) may be determined. As discussed herein, this and other determinations/calculations may be performed by a computational portion (e.g., computational portion 105) that is in communication with light source 351, radiation detector 353, and/or other components. Components for determining other characteristics of a blood sample may be used at other analysis portions of chip 303.

In some embodiments, methods for minimally invasive measurement of ABG values are provided. FIG. 4 illustrates a process 400, which is an example of a process for obtaining and using minimally invasive measurement of ABG values. Process 400 may include an operation 401, wherein a minimally invasive sample collection apparatus is applied or otherwise engaged with a tissue of a patient to obtain a blood sample therefrom. For example, an apparatus similar to those illustrated in FIGS. 2 and 3A having a micro needle or cannula may be used to prick the skin of a patient and obtain a capillary (via a capillary rich tissue) or venous (via a vein) blood sample of a patient. In some embodiments, a small amount of blood (e.g., 15-20 μl) is obtained for analysis (about 5-10 μl of which may be used in each individual analysis portion).

In some embodiments, the tissue of the patient may be pre-treated before the blood sample is obtained. For example, a tissue of the patient may be warmed prior to obtaining a sample. Warming the tissue may cause vasodilation of the vessels from which blood is obtained and therefore may provide blood characteristics that more closely resemble arterial blood measurements. For example, the heel of an infant may be warmed prior to obtaining a blood sample for ABG measurements from the infant. Another example may include applying vasodilator chemicals to the heel of an infant or other patient.

In an operation 403, the blood sample is separated into a plurality of analysis portions of the minimally invasive collection apparatus (e.g., analyte separation portions 205a-205n of FIG. 2; branch channels 309a-309n and analysis portions 311a-311n of FIG. 3). In some implementations, only a single analysis portion maybe used (e.g., when multiple characteristics can be measured in a single analysis portion or wherein only a single characteristic is to be obtained). In some embodiments, the blood sample is obtained from the patient and separated into the plurality of analysis portions without exposing (or minimally exposing) the blood sample to oxygen, air, or other reactive substances. For example, as discussed herein, the collection apparatus may be filled with an inert gas, may have a vacuum therein, and/or may have dimensions that minimally expose the blood sample to error causing substances (e.g., oxygen, air, or other reactive substances).

In an operation 405, one or more characteristics of the blood sample are obtained (e.g., using measurement components as described herein with respect to FIGS. 1, 2 and 3B). For example, in some embodiments, one or more of a CO₂ measurement, an 0₂ measurement, and/or a pH measurement. Other measurements may also be obtained such as, for example, glucose-6-phosphate dehydrogenase (G6PD) deficiency measurements, jaundice measurements (e.g., bilirubin levels), and/or other measurements. In some embodiments, the one or more characteristics may be calculated/determined/derived at a computational portion (e.g., computational portion 105 and/or one or more modules 107a-107n thereof) from signals sent by analysis components. In some embodiments, the one or more characteristics may be calculated/determined/derived (e.g. using processors and logic integrated with a spectrograph/radiation detectors or other analysis components) and sent to a computational portion (e.g., computational portion 105 and/or one or more modules 107a-107n thereof).

In an operation 407, the one or more characteristics of the blood sample may be used to derive ABG measurements. The ABG measurements may include O₂ concentration, CO₂ concentration, blood pH, and/or other characteristics. In some embodiments, a function or correlation graph may be used to convert the measured sample characteristics (e.g., O₂, CO₂, pH, etc.) into ABG values. In some embodiments, additional information may be used with determined sample characteristics to derive ABG values. For example, in some embodiments, the type of blood or location of blood draw may be used with sample characteristics to derive ABG values. For instance, capillary blood may be sampled (i.e., from a patient's capillaries) and a function or correlation graph specifically intended for use in converting capillary blood samples to ABG values may be used. According to many studies, the arterialization of capillary blood is linearly related with arterial blood gas values. In another example, a function or correlation graph specifically intended for use in converting venous blood samples into ABG values may be used when venous blood is used for a blood sample. Other types of information may also be used to select functions or correlation graphs for converting sample values to ABG values such as, for example, patient age, physical condition of a patient (e.g., healthy, hypothermic, etc.), pathology information relating to a patient (e.g., hypoxemia, metabolic acidosis, respiratory alkylosis, etc.), and/or other information. In some embodiments, a function or correlation graph used to convert sampled non-arterial blood into arterial values may be constructed by plotting a calibration curve between a spectrogram of sampled blood characteristics (e.g., O₂, CO₂, etc.) and arterial blood gas values using a gold standard such as, for example, arterial blood samples obtained using oxygen and carbon dioxide electrodes. This calibration curve may be stored as a look up table (e.g., in computational system 105) and used to derive ABG values from sampled characteristics.

In an operation 409, the derived ABG measurements may be used, alone or with other data, to assess the condition of a patient, to assess the results or effectiveness of a therapy, and/or otherwise used. For example, arterial O₂, CO₂, and/or pH values may be useful in assessing the health of a patient. In another example, the ABG values may be used to assess whether ventilation or other respiratory therapy is effective in achieving predetermined goals (e.g., a specific arterial O₂ concentration, etc.).

In some implementations, the ABG values may be used as part of closed-loop respiratory therapy (e.g., fraction of inspired oxygen (FiO₂) management). Using the minimally invasive devices and methods provided herein, clinicians can arrive at ABG values using a very small volume of blood (obtained with minimal invasive interaction with the patient). These ABG values can, in turn, be used to help clinicians in choosing ventilation strategies and other courses of action (FiO₂ management is one of those strategies). FIG. 5 illustrates a method 500, which is an example of a method for closed loop integration of ABG values into respiratory therapy management. In an operation 501, ABG values 551, patient history and assessment data 553, respiratory monitoring values 555 (e.g., saturation of peripheral oxygen—SpO₂), end-tidal CO₂ values 557, and/or other information 559 may be received/determined In some embodiments, these parameters may be received and/or calculated by a CDS engine (a rule-based clinical decision support engine) which may be one of the one or more modules 107a-107n discussed herein.

In an operation 503, the information from operation 501 may be used to formulate ventilation or other respiratory therapy parameters for a patient. For example, the information may be used to determine whether a patient is adequately ventilated or not. If the patient is not adequately ventilated, a ventilator setting can be changed or other actions can be taken. In an operation 505, these parameters may be communicated to a respirator or other apparatus for providing respiratory therapy such that the respiratory therapy is provided to a patient by the apparatus in accordance with the apparatus.

In an operation 507, one or more values/measurements may be determined/made after or during delivery of treatment. In some embodiments, these values may include ABG values, patient assessment data, respiratory monitoring values (e.g., saturation of peripheral oxygen—SpO₂), end-tidal CO₂ values, and/or other information. In an operation 509, the values/measurements may be used to formulate additional respiratory therapy parameters for further treatment. Process 500 may then return to operation 505, wherein respiratory therapy is provided to the patient based on the parameters. In this manner, a closed-loop system is provided.

In some embodiments, tangible computer-readable media comprising computer-executable instructions for causing one or more computer processors (e.g., processors 109) to perform one or more of the features and functions set forth herein, including the operations of the methods described herein, may be provided.

The systems described herein are exemplary system configurations. Other configurations may exist. Those having skill in the art will appreciate that the invention described herein may work with various configurations. Accordingly, more or less of the aforementioned system components may be used and/or combined in various embodiments. It should also be understood that various software modules that are utilized to accomplish the functionalities described herein may be maintained on different components than computational system 105, as desired or necessary. In other embodiments, as would be appreciated, the functionalities described herein may be implemented in various combinations of hardware and/or firmware, in addition to, or instead of, software. Furthermore, various operations of the methods described herein, while described in a particular order, may be performed in different orders as would be appreciated by those having skill in the art. In some embodiments, more of less of the described operations may be used.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims For example, it is to be understood that the present invention contemplates that, to the extent possible, one or more features of any embodiment can be combined with one or more features of any other embodiment.

1. A system for providing arterial blood gas measurements, comprising:
a sample collection portion positioned in contact with a tissue of the patient such that a blood sample travels from the tissue of the patient into the sample collection portion without being exposed to an environment outside of the sample collection portion;
one or more analysis portions in fluid communication with the sample collection portion, wherein each of the one or more analysis portions analyze one or more characteristics of the blood sample; and
at least one processor configured to:
receive the one or more characteristics of the blood sample, and
calculate one or more arterial blood gas measurements using the one or more characteristics.

2. The system of claim 1, wherein the at least one processor is further configured to receive information relating to the circumstances surrounding collection of the blood sample, and wherein calculating one or more arterial blood gas measurements further uses the information relating to the circumstances surrounding the collection of the blood sample.

3. The system of claim 2, wherein the one or more circumstances surrounding collection of the blood sample include a type of blood sample, and wherein calculation of one or more arterial blood gas measurements further includes selecting a function configured for analyzing blood samples of the received type, the calculation of the one or more arterial blood gas measurements using the selected function.

4. The system of claim 1, wherein one or more arterial blood gas measurements are input into a closed loop system for providing respiratory therapy to the patient.

5. The system of claim 1, wherein one or more of the sample collection portion or the one or more analysis portions include one or more microtubules or microchannels.

6. A method for providing arterial blood gas measurements, comprising:
positioning a sample collection portion in contact with a tissue of the patient such that a blood sample travels from the tissue of the patient into the sample collection portion without being exposed to an environment outside of the sample collection portion, and wherein the blood sample travels to one or more analysis portions in fluid communication with the sample collection portion, each of the one or more analysis portions analyzing one or more characteristics of the blood sample;
receiving at one or more processors of a computational portion the one or more characteristics of the blood sample; and
calculating one or more arterial blood gas measurements using the one or more characteristics.

7. The method of claim 6, further comprising receiving information relating to the circumstances surrounding collection of the blood sample, and wherein calculating one or more arterial blood gas measurements further uses the information relating to the circumstances surrounding the collection of the blood sample.

8. The method of claim 7, wherein the one or more circumstances surrounding collection of the blood sample include a type of blood sample, and wherein calculating one or more arterial blood gas measurements further includes selecting a function configured for analyzing blood samples of the received type, the calculation of the one or more arterial blood gas measurements using the selected function.

9. The method of claim 6, further comprising, inputting the one or more arterial blood gas measurements into a closed loop system for providing respiratory therapy to the patient.

10. The method of claim 6, wherein one or more of the sample collection portion or the one or more analysis portions include one or more microtubules or microchannels.

11. A system for providing arterial blood gas measurements, comprising:
sample collection means positioned in contact with a tissue of the patient such that a blood sample travels from the tissue of the patient into the sample collection means without being exposed to an environment outside of the sample collection means;
one or more analysis means in fluid communication with the sample collection means for analyzing one or more characteristics of the blood sample; and
processing means configured to:
receive the one or more characteristics of the blood sample, and
calculate one or more arterial blood gas measurements using the one or more characteristics.

12. The system of claim 11, the processing means being further configured to receive information relating to the circumstances surrounding collection of the blood sample, and wherein calculating one or more arterial blood gas measurements further uses the information relating to the circumstances surrounding the collection of the blood sample.

13. The system of claim 12, wherein the one or more circumstances surrounding collection of the blood sample include a type of blood sample, and wherein calculation of one or more arterial blood gas measurements further includes selecting a function configured for analyzing blood samples of the received type, the calculation of the one or more arterial blood gas measurements using the selected function.

14. The system of claim 11, wherein one or more arterial blood gas measurements are input into a closed loop system for providing respiratory therapy to the patient.

15. The system of claim 11, wherein one or more of the sample collection means or the one or more analysis means include one or more microtubules or microchannels.
